(19) United States
(12) Patent Application Publication
(10) Pub. No.: US 2018/0098510 A1
(43) Pub. Date: Apr. 12, 2018

(54) METHOD OF HARVESTING CRUCIFEROUS SPROUTS FOR INCREASING THE AMOUNT OF GLUCOSINOLATE IN CRUCIFEROUS SPROUTS

(71) Applicant: KOREA INSTITUTE OF SCIENCE AND TECHNOLOGY, Seoul (KR)

(72) Inventors: Hyoungseok KIM, Seoul (KR); Eunok KIM, Seoul (KR); Sangrok OH, Gangneung-si (KR); Chuwon NHO, Seoul (KR); Juyeon HONG, Gangneung-si (KR); Sunyoung KIM, Seoul (KR)

(73) Assignee: KOREA INSTITUTE OF SCIENCE AND TECHNOLOGY, Seoul (KR)

(21) Appl. No.: 15/725,903

(22) Filed: Oct. 5, 2017

(30) Foreign Application Priority Data
Oct. 6, 2016 (KR) 10-2016-0129014

(51) Int. Cl. A01G 7/04; A01G 1/00; A61K 36/31; H05B 33/08

(52) U.S. Cl. CPC A01G 7/045; H05B 33/0803; A61K 36/31; A01G 1/001

(57) ABSTRACT

A method for growing cruciferous sprout uses light emitted diode (LED) as a light source in growing the cruciferous sprout, which consequently increases the amount of the glucosinolates of the cruciferous sprout. The cruciferous sprout with increased anticancer activity can be grown with the easy light-controllable, eco-friendly and economical method.

### BACKGROUND OF THE INVENTION

### 1. Field of the Invention

The present invention relates to method of harvesting(growing) cruciferous sprouts for increasing the amount of glucosinolates in cruciferous sprouts. More specifically, the present invention relates to the methods of growing cruciferous sprouts using LED light of specific wavelength range as light sources to increase the amount of glucosinolates in cruciferous sprouts.

### 2. Description of the Related Art

The glucosinolates is secondary metabolite of cruciferous vegetables such like mustard, horseradish, cabbage, daikon, cauliflower, kale, broccoli, and Chinese cabbage and is converted to isothiocyanate by myrosinase of hydrolase when cutting, chewing or digesting the cruciferous vegetables.

In intestine, the glucosinolates is decomposed into isothiocyanate by microorganism. The glucosinolates is known as anticancer substance and especially effective for the breast cancer, bladder cancer, liver cancer and so on.

The glucosinolates can adjust white blood cell and cytokine very well and has enzymes for restricting the growth of the tumor in breast, liver, large intestine, lung, throat, and stomach.

Prior art document 1 (KR1525143B1) relates to method of growing the daikon sprout with excellent antioxidant activity and discloses how to treat the jasmonic acid or methyl jasmonate in daikon sprout and to increase the amount of the glucosinolates. Prior art document 2 (KR1638205B1) discloses novel method to extract the glucosinolates from broccoli sprout or seed and to refine it.

Above mentioned prior arts is to increase the amount of the glucosinolates of the sprout based on the chemical treatment of the jasmonic acid, methyl jasmonate or the like. Therefore, those are different from the present invention which increases the amount of the glucosinolates with light source control.

### SUMMARY OF THE INVENTION

An object of the present invention is to provide the method of growing cruciferous sprout with increased anticancer activity and therefor provide another improved solution for the conventional arts.

Also, the present invention aims to provide the easy light-controllable, eco-friendly and economical method of growing cruciferous sprout.

In order to achieve the above object, in accordance with one aspect of the present invention, method for growing cruciferous sprout, comprises using LED as light source to increase the amount of the glucosinolates of the cruciferous sprout in growing the cruciferous sprout.

Here, the cruciferous sprout is red cabbage and the LED emits the light of the wavelength within specific range to increase the amount of the glucosinolates of the cruciferous sprout.

Also, the specific range would be 380˜550 nm, especially 450˜550 nm.

In addition, the glucosinolates is mixture of at least one selected from a group comprising glucoiberin, progoitrin, glucoraphanin, sinigrin, gluconapin, glucobrassicin, gluconasturtiin and neoglucobrassicin.

As stated above, according to the present invention, the cruciferous sprout with increased anticancer activity can be grown with the easy light-controllable, eco-friendly and economical method.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 shows the pictures of the sprouts according to comparative example 1 and comparative example 2 of the present invention.

FIG. 2 shows the pictures of the sprouts according to embodiment 1˜8 of the present invention.

FIG. 3 is a graph showing the hypocotyl length of the sprouts of the comparative examples and embodiments according to the present invention.

FIG. 4 is a graph showing the production yield of the sprouts of the comparative examples and embodiments according to the present invention.

### DETAILED DESCRIPTION OF THE INVENTION

Hereinafter, with reference to the accompanying drawings, preferred embodiments of the present invention will be described in detail. However, the explanation on the known functions and configurations that may obscure the subject matter of the present invention from the detailed description of the following description and from the accompanying drawings will be omitted. In addition, the same components throughout the drawings are referred to by the same reference numerals as possible which is to be noted.

The terms used in this specification and claims is not to be construed as limited to dictionary meanings, but can be defined and interpreted based on the meanings and concepts corresponding to technical aspects of the present invention in the principle that inventors define the terms appropriate to the concept of a term to describe his own invention in the best way. Therefore, the present embodiment and the configuration shown in the drawings and described in the specification is merely nothing but a preferable embodiment of the present invention, as not intended to represent all the technical concept of the present invention, so that it should be understood that many equivalents and varied modified embodiments of the present invention that can be made in the present application point.

The present invention relates to the method of growing cruciferous sprouts using LED of specific wavelength range as light sources to increase the amount of glucosinolates in cruciferous sprouts.

Most of the cruciferous vegetables are herbaceous plants and economically important. Its leaf has zippy tang and its flower of white, yellow, or light purple has four petals and sepals which resemble a cross. Most important genus is brassica which comprises old world vegetable of 50 species such like cabbage, mustard and rapa. It comprises another edible vegetables such like kohlrabi, brassica fimbriata, napobrassica, and brassica rapa. In Korea, the cruciferous vegetables of 18 genera and 50 species grow mainly in fields or mountains. Recently, exotic cruciferous vegetables are planted in flowerpot or garden.

With LED lamp of specific wavelength range as light source, we have done the experiment to find a method of increasing the glucosinolates and quinone reductase(QR) activity, which is that of growing the improved cruciferous vegetable according to the present invention.

As light source, the LED lamp has many advantages of mercuryfree eco-friendly, low power consuming, pulse-shape lighting, and light quality and power easy-controllable lamp.

The glucosinolates shows various activities, especially cancer preventive activity, in human body. The cruciferous vegetables are well known to contain lots of the glucosinolates and various attempts to increase the amount of the glucosinolates of the cruciferous sprouts have been done. The present invention specifies conditions of the light source to increase the amount of the glucosinolates and therefore to get excellent activities of functional materials.

Through below embodiments, the present invention will be understood in detail. It will be apparent to those of ordinary skill in the art that the scope of the present invention is not limited by the embodiments which are disclosed for the clarification of the present invention.

### EMBODIMENT

### Experimental Conditions

1. Vegetable species
Red cabbage

2. Seed weight
Red cabbage (3 g/cell)*3 repetition

3. Seed disinfection(clean and soak)
Input calcium hypochlorite 10 mL per seed 1 g and proceed seed for 15 minutes, and then clean it with tap water and soak it in distilled water for 4 hours

4. Sowing the seed
Sow the seed in grow plastic container.

5. Light treatment
Darkening treatment : 2 days, the blind and the foil cover
Brightening treatment: 5 days, intensity 50 umol (except for 780 nm)
Grow temperature, relative humidity: 23±2° C(25/19), 45±5%

6. Water provision
Pouring water of 150 mL into each of the grow plastic container evenly every day and spraying water 3˜4 times/day.
Changing the distilled water of the grow plastic container every 2 days.

7. The others
Changing the place of the grow plastic container during the growth.

### COMPARATIVE EXAMPLES 1˜2 AND EMBODIMENTS 1˜8

Comparative example 1 : using fluorescent light as light source
Comparative example 2 : using 3 color LED as light source
Embodiment 1 : using 385 nm LED
Embodiment 2 : using 450 nm LED
Embodiment 3 : using 465 nm LED
Embodiment 4 : using 520 nm LED
Embodiment 5 : using 620 nm LED
Embodiment 6 : using 645 nm LED
Embodiment 7 : using 660 nm LED
Embodiment 8 : using 780 nm LED

### Experimental Results

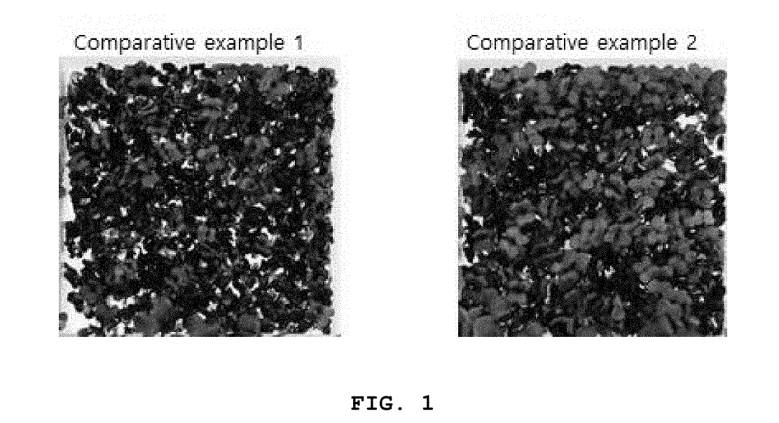

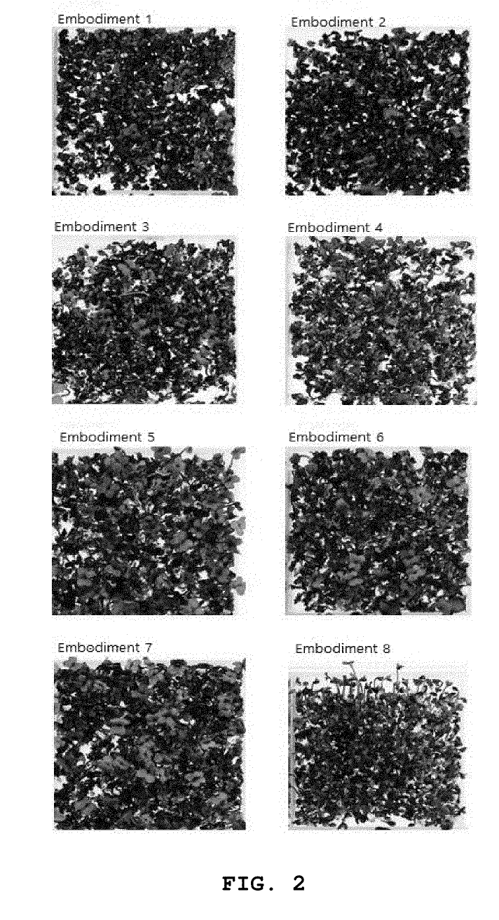

FIG. 1 shows the pictures of the sprouts according to comparative example 1 and comparative example 2 of the present invention and FIG. 2 shows the pictures of the sprouts according to embodiment 1˜8 of the present invention.

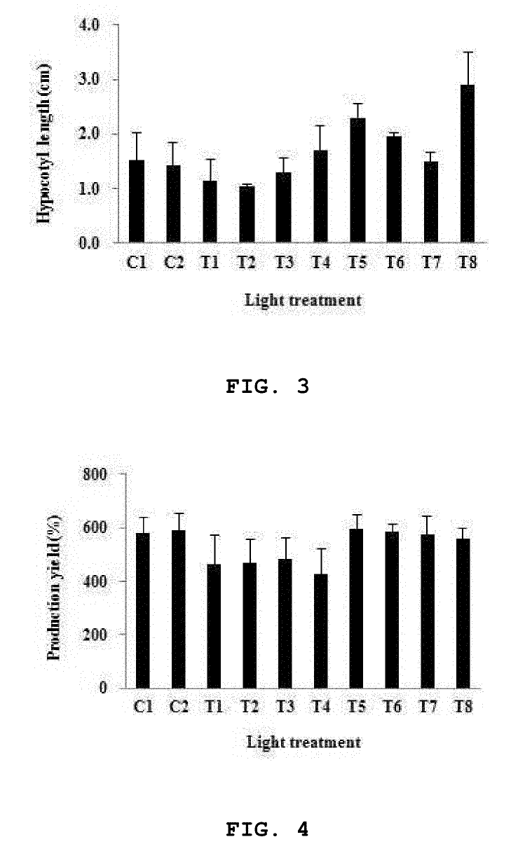

FIG. 3 is a graph showing the hypocotyl length of the sprouts of the comparative examples and embodiments according to the present invention and FIG. 4 is a graph showing the production yield of the sprouts of the comparative examples and embodiments according to the present invention(C1: comparative example 1, C2: Comparative example 2, T1: Embodiment 1, T2: Embodiment 2, T3: Embodiment 3, T4: Embodiment 4, T5: Embodiment 5, T6: Embodiment 6, T7: Embodiment 7, T8: Embodiment 8).

The growth and development states are recognized from the picture and graph of FIG. 1˜4 and the form of the vegetable would be a criterion of the preference of the consumer for the sprout.

As noted from the FIGS, the wavelength of the embodiments 1˜4 induces the accumulation of the anthocyanin of the red cabbage sprout to add red color and the length of the sprout in the embodiments 1˜4 is similar to that of the sprout sold in market place. Therefore, the method to grow the preferred sprout is provided.

The results from the above grow experiments are like the below tables 1,2.

The table 1 represents the amount of the glucosinolates according to each of the comparative examples and embodiments.

TABLE 1

| | Glucosinolate content (M/g DW) | | | | |
|---|---|---|---|---|---|
| | glucoiberin | Progoitrin | glucoraphanin | sinigrin | gluconapin |
| Comparative | 3.11 ± 0.32 | 15.88 ± 0.92 | 7.67 ± 0.84 | 6.85 ± 0.10 | 1.67 ± 0.04 |
| example 1 | | | | | |
| Comparative | 3.02 ± 0.81 | 14.82 ± 2.01 | 7.80 ± 1.98 | 6.50 ± 0.73 | 1.47 ± 0.19 |
| example 2 | | | | | |
| Embodiment 1 | 3.27 ± 0.15 | 20.02 ± 1.28 | 7.21 ± 1.00 | 8.59 ± 1.80 | 1.77 ± 0.13 |
| Embodiment 2 | 3.48 ± 0.53 | 17.03 ± 2.84 | 7.96 ± 0.44 | 8.05 ± 2.60 | 1.74 ± 0.35 |
| Embodiment 3 | 3.12 ± 0.76 | 19.27 ± 0.35 | 7.65 ± 2.49 | 8.19 ± 0.99 | 1.87 ± 0.18 |
| Embodiment 4 | 3.21 ± 0.35 | 19.25 ± 2.11 | 7.08 ± 0.88 | 8.38 ± 2.04 | 1.80 ± 0.12 |
| Embodiment 5 | 3.29 ± 0.31 | 15.07 ± 0.16 | 8.11 ± 0.81 | 6.65 ± 0.23 | 1.55 ± 0.12 |
| Embodiment 6 | 3.23 ± 0.28 | 14.65 ± 1.30 | 7.99 ± 0.81 | 6.38 ± 0.39 | 1.51 ± 0.06 |
| Embodiment 7 | 2.98 ± 0.42 | 13.73 ± 0.91 | 7.46 ± 1.01 | 6.12 ± 0.18 | 1.44 ± 0.08 |
| Embodiment 8 | 2.94 ± 0.23 | 14.31 ± 0.96 | 7.29 ± 0.49 | 6.52 ± 0.19 | 1.68 ± 0.10 |

| | Glucosinolate content (M/g DW) | | | |
|---|---|---|---|---|
| | glucobrassicin | gluconasturtiin | neoglucobrassicin | total |
| Comparative | 1.46 ± 0.17 | 1.47 ± 0.21 | 0.58 ± 0.06 | 38.71 ± 0.47 |
| example 1 | | | | |
| Comparative | 1.59 ± 0.32 | 1.74 ± 0.44 | 0.51 ± 0.13 | 37.46 ± 6.13 |
| example 2 | | | | |
| Embodiment 1 | 1.29 ± 0.32 | 1.76 ± 0.33 | 0.78 ± 0.04 | 44.69 ± 2.21 |
| Embodiment 2 | 1.19 ± 0.06 | 1.44 ± 0.52 | 0.59 ± 0.17 | 41.18 ± 6.69 |
| Embodiment 3 | 1.23 ± 0.20 | 1.36 ± 0.33 | 0.66 ± 0.07 | 43.35 ± 2.08 |
| Embodiment 4 | 1.09 ± 0.11 | 1.65 ± 0.26 | 0.71 ± 0.04 | 43.17 ± 4.22 |
| Embodiment 5 | 1.49 ± 0.23 | 1.72 ± 0.22 | 0.69 ± 0.06 | 38.56 ± 0.86 |
| Embodiment 6 | 1.32 ± 0.08 | 1.56 ± 0.43 | 0.61 ± 0.11 | 37.24 ± 2.99 |
| Embodiment 7 | 1.42 ± 0.05 | 1.45 ± 0.11 | 0.54 ± 0.03 | 35.15 ± 2.33 |
| Embodiment 8 | 0.76 ± 0.06 | 1.40 ± 0.16 | 0.64 ± 0.06 | 35.53 ± 1.86 |

The table 2 represents the inducted activity of Quinone Reductase(QR) according to each of the comparative examples and embodiments.

TABLE 2

| | IC₅₀ | CD | CI |
|---|---|---|---|
| Comparative example 1 | 33.53 | 54.27 | 0.62 |
| Comparative example 2 | 30.23 | 29.41 | 1.03 |
| Embodiment 1 | 39.50 | 11.87 | 3.33 |
| Embodiment 2 | 82.58 | 7.27 | 11.36 |
| Embodiment 3 | 21.47 | 2.81 | 7.63 |
| Embodiment 4 | 20.55 | 3.53 | 5.83 |
| Embodiment 5 | 43.81 | 78.57 | 0.56 |
| Embodiment 6 | 33.12 | 200.00 | 0.17 |
| Embodiment 7 | 2.25 | 64.21 | 0.03 |
| Embodiment 8 | 5.80 | 73.37 | 0.08 |

* CD: Mean value of the concentration required to double the specific activity of QR
* IC₅₀: Mean value of the half-maximal inhibitory concentration of cell vialbility
* CI: Chemoprevention Index: ratio between IC₅₀ and CD

As noted from the experimental results, it is in the wavelength range including the embodiment 1 (385 nm), the embodiment 2 (450 nm), the embodiment 3 (464 nm), and the embodiment 4 (520 nm) that the amount of the glucosinolates are increased comparative to the other embodiments and the activity of QR is high.

The below table 3 represents increasing ratio of each kind of the glucosinolates in each embodiment in comparison with mean value of that in the comparative examples 1, 2.

TABLE 3

| | Glucosinolate content (M/g DW) | | | | | | | | |
|---|---|---|---|---|---|---|---|---|---|
| | glucoiberin | Progoitrin | glucoraphanin | sinigrin | gluconapin | glucobrassicin | gluconasturtiin | neoglucobrassicin | total |
| Embodiment 1 | 6.7% | 30.4% | −6.8% | 28.7% | 12.7% | −15.4% | 9.7% | 43.1% | 17.3% |
| Embodiment 2 | 13.5% | 10.9% | 2.9% | 20.6% | 10.8% | −22.0% | −10.3% | 8.3% | 8.9% |
| Embodiment 3 | 1.8% | 25.5% | −1.1% | 22.7% | 19.1% | −19.3% | −15.3% | 21.1% | 13.8% |
| Embodiment 4 | 4.7% | 25.4% | −8.5% | 25.5% | 14.6% | −28.5% | 2.8% | 30.3% | 13.4% |
| Embodiment 5 | 7.3% | −1.8% | 4.8% | −0.4% | −1.3% | −2.3% | 7.2% | 26.6% | 1.2% |
| Embodiment 6 | 5.4% | −4.6% | 3.3% | −4.4% | −3.8% | −13.4% | −2.8% | 11.9% | −2.2% |
| Embodiment 7 | −2.8% | −10.6% | −3.6% | −8.3% | −8.3% | −6.9% | −9.7% | −0.9% | −7.7% |
| Embodiment 8 | −4.1% | −6.8% | −5.8% | −2.3% | 7.0% | −50.2% | −12.8% | 17.4% | −6.7% |

As noted from the table 3, the increasing ratios of the total amount of the glucosinolates in the embodiments 1˜4 are more than 8.9%, which are certainly high increases. Especially, the increase of the amount of the progoitrin, the sinigrin, and the neoglucobrassicin is very high

Implementations of the various techniques described herein area digital electronic circuitry, or computer hardware, firmware, software, or may be implemented in a combination of them. Implementations can be implemented by a data processing device, for example, a programmable processor, a computer, or for processing by the operation of a plurality of computers, or to control the operation, the computer program product, i.e. the information carrier, for example, machine-readable apparatus (computer readable medium) or a radio signal. The computer program as stated above can be recorded in a programming language of any type, including a substituted or interpret compiled language, as a stand-alone program or as a module, component, subroutine, or in the computing environment, it may be deployed in any form, including as appropriate, including the use of other units. Computer program can be distributed across one or more computer or a number of sites to be processed on multiple computers at one site, and can be connected by a communication network.

Processors suitable for the processing of the computer program comprise as an example, includes both general and special purpose microprocessors, and more than one processors of any kind of digital computer. Generally, a processor may receive commands or data from read-only memory or random-access memory or both. The computer can include more than one memory device saving at least one processor and commands and data which executes commands. For example, it includes magnetism, magnetic-optical disks, or optical disks, or transmitting this data or combining both, or it can receive or transmit data or combine both. Information carriers appropriate for specifying computer program commands or data as an example, semiconductor memory device, for example, includes hard disks, floppy disks, and magnetic tape, such as magnetic media, CD-ROM (Compact Disk Read Only Memory), DVD (Digital Video disk) and the like optical recording media, floptical disk, such as magneto-optical media, ROM (Read Only Memory), RAM (Random Access memory), comprises a flash memory, EPROM (Erasable Programmable ROM), EEPROM (Electrically Erasable Programmable ROM) etc. Processor and memory can be added or included by special purpose logic circuitry.

The present description herein includes details a number of specific implementations, but it cannot be understood as limited for any invention or scope for patent claims, rather to be understand as explanation about featuring specific implementation of specific invention. The specific features of the present description in context of each implementation herein can be implemented in combination in a single embodiment. Conversely, it also can be implemented in a plurality of embodiments with different features, any suitable sub-combination or separately described in the context of a single embodiment. Furthermore, the features can be combined as specific combinations or described as claimed in early, but one or more features from claimed combinations can be excluded from the combination in some cases, the claimed combination can be changed as sub-combination or its modifications.

Likewise, although it describes operations as particular order, it cannot be understood that performing those operations as the specific or sequential order described to achieve desired results or being performed for all described operations. In certain case, multi-tasking and parallel processing can be advantageous. In addition, separation of various system components in the embodiments described above should not be understood to require in any embodiment such a separation, the described program components and systems are generally integrated together in a single software product or be packaged into multiple software products number that should be understood.

On the other hand, the embodiments of the invention disclosed in the specification and drawings are not presented merely a specific example for clarity and are not intended to limit the scope of the invention. It addition to the embodiments disclosed herein another modification based on the technical ideas of the invention are possible embodiments, it will be apparent to those of ordinary skill in the art.

What is claimed is:

1. Method for growing cruciferous sprout, comprising:
using LED as light source to increase the amount of the glucosinolates of the cruciferous sprout in growing the cruciferous sprout.

2. The method according to claim 1,
wherein the cruciferous sprout is red cabbage.

3. The method according to claim 2,
wherein the LED emits the light of the wavelength within specific range to increase the amount of the glucosinolates of the cruciferous sprout.

4. The method according to claim 3,
wherein the specific range is 380˜550 nm.

5. The method according to claim 4,
wherein the specific range is 450˜550 nm.

6. The method according to claim 1,
wherein the glucosinolates is mixture of at least one selected from a group comprising glucoiberin, progoitrin, glucoraphanin, sinigrin, gluconapin, glucobrassicin, gluconasturtiin and neoglucobrassicin.
